(19) United States
(12) Patent Application Publication
(10) Pub. No.: US 2023/0227832 A1
(43) Pub. Date: Jul. 20, 2023

(54) ANTICANCER APTAMERS AND USES THEREOF

(71) Applicant: CONSIGLIO NAZIONALE DELLE RICERCHE, Roma (RM) (IT)

(72) Inventors: Vittorio DE FRANCISCIS, Rome (RM) (IT); Gerolama CONDORELLI, Rome (RM) (IT); Silvia CATUOGNO, Rome (RM) (IT); Carla Lucia ESPOSITO, Rome (RM) (IT); Cristina QUINTAVALLE, Rome (RM) (IT); Francesco INGENITO, Rome (RM) (IT)

(73) Assignee: CONSIGLIO NAZIONALE DELLE RICERCHE, Roma (RM) (IT)

(21) Appl. No.: 18/002,213

(22) PCT Filed: Jun. 17, 2021
(86) PCT No.: PCT/EP2021/066448

§ 371 Date: Dec. 16, 2022

(30) Foreign Application Priority Data
Jun. 18, 2020 (IT) 102020000014671

(51) Int. Cl. C12N 15/115; C12N 15/10; C12Q 1/6886

(52) U.S. Cl. CPC C12N 15/115; C12N 15/1048; C12Q 1/6886; C12N 2310/16

(57) ABSTRACT

The present invention relates to a nucleotide aptamer or a variant thereof, or a functional fragment thereof, the medical or diagnostic use thereof, the related pharmaceutical composition and a method for selecting a nucleotide aptamer which specifically binds to exosomes isolated from target cells. The present invention further relates to a kit and nucleic acid coding for the aptamer.

### FIELD OF THE INVENTION

The present invention relates to a nucleotide aptamer or a variant thereof, or a functional fragment thereof, the medical or diagnostic use thereof, the related pharmaceutical composition and a method for selecting a nucleotide aptamer which specifically binds to exosomes isolated from target cells. The present invention further relates to a kit and nucleic acid coding for the aptamer.

### PRIOR ART

Breast cancer (BC) is the most common type of cancer and a leading cause of death in women, with more than 450,000 women dead/year (Harbeck N, Gnant M. Lancet. 2017; Rugo H S et al., N Engl J Med. 2016). The stage of the disease at diagnosis strongly influences patient survival. The effectiveness of the diagnostic strategies available, such as mammography, is limited by the minimal tumor size required for visualization. The search for circulating markers is a fundamental challenge to be able to identify the tumor early, in a non-invasive and cost-effective manner (Joyce D P et al. J, Cancer. 2016). At the same time, there is a need to develop new therapeutic options which are safe and effective.

In recent years, extracellular vesicles derived from endosomes (exosomes) are emerging as promising biomarkers and therapeutic targets in cancer. Exosomes are vesicles with a diameter of 50-250 nm, released into circulation by most cells and containing nucleic acids and proteins (Hannafon B N, Ding W Q., Int J Mol Sci. 2013). Tumor cells, including BC cells, have been shown to release large amounts of exosomes which, being stable and easily accessible in biological fluids, can be used for tumor-specific detection (Lowry M C, Clin Chem. 2015; Chia B S et al. Trends Anal. Chem 2017). In addition, several studies have shown that exosomes represent an important mechanism of communication between tumor cells and the surrounding stroma affecting tumor growth and progression, therapy resistance, and immune surveillance (Mohammed H et al., Int J Mol Sci. 2017; Hoshino A et al., Nature. 2015; Donnarumma E et al., Oncotarget. 2017; Bliss S A et al., Can Res. 2016; Huang M B et al., Oncotarget. 2017). To date, several enriched proteins in exosomes have been characterized, such as members of the tetraspanin family (CD9, CD63 and CD81), components of endosomal sorting complexes (TSG101 and Alix) and heat-shock proteins (Hsp60, Hsp70 and Hsp90). (Taylor D D et al., Immunopathol. 2011). Some exosomal proteins (i.e., HER2, CD47, Del-1), microRNAs (i.e., miR-1246, miR-21) and RNAs (i.e., NANOG, NEUROD1, HTR7) have also been associated with BC recurrence, metastasis formation and patient survival, confirming the theranostic value of these vesicles (Wang M et al. Clin, Transl Oncol. 2018). Additionally, several protocols for the detection of exosomes are emerging in the literature (Cheng N et al., Trends Biotechnol. 2019). However, despite these advances, tools for the specific recognition of tumor exosomes are still lacking.

Aptamers are a promising class of specific recognition molecules. These are nucleic acids capable of binding high affinity molecular targets and have many advantages for both diagnostic and therapeutic applications such as low toxicity, easy modification and lack of immunogenicity (Wu X et al., Theranostics. 2015). Aptamers are selected through an in vitro procedure called SELEX (Systematic Evolution of Ligands by Exponential Enrichment) which has been successfully applied to a wide range of targets from small molecules to complex systems such as cells and tissues (Catuogno S et al., Biomedicine 2017). The high recognition specificity and flexibility of aptamers has allowed the use thereof in numerous applications for the diagnosis, therapy and discovery of biomarkers (Maimaitiyiming Y, et al. J, Cancer Res Clin Oncol. 2019).

The following documents disclose aptamers selection and their use for the identification of cancer exosomes: Quintavalle et al. (Annals of Oncology 2019); Diaz-Fernandez Ana et al. (Anal Chimica Acta 2020); An Yu et al. (Anal Chem 2020); Hassan Eman et al. (Trac Trend in Anal Chem 2020); Ringrong Huang et al. (Small 2019); Hornung Tassilo et al. (Nucleic Acids Res 2020) and WO2014100434. Lyu et al. (Theranostics, 2016) provides an overview on cell-SELEX procedure for aptamer selection.

Zhou Y et al., (Analyst. 2019 Dec. 16; 145(1):107-114. doi: 10.1039/c9an01653h) underline the importance of developing tools for targeting exosomes and describe a proof of principle of the possibility of using aptamers to detect them, using already-published molecules against generic targets.

Zou J et al., (Anal Chem. 2019 Feb. 5; 91(3):2425-2430. doi: 10.1021/acs.analchem.8b05204) describe the use of exosomes as a container for directing drugs to neoplastic cells. To this end, the exosomes are conjugated to an aptamer capable of recognizing neoplastic cells and thus allowing the accumulation thereof in such cells.

Therefore, methodologies should be developed to identify molecules able of specifically recognizing exosomes derived from target cells, in particular cancer cells such as BC and relative molecules for therapeutic and diagnostic use.

### SUMMARY OF THE INVENTION

Exosomes are ideal candidates for the development of early and non-invasive diagnostic strategies and innovative therapeutic approaches for different types of cancer, including BC. Although exosome-enriched proteins have been characterized, tools which can distinguish tumor exosomes in a highly specific manner are not currently available and represent a key element for the clinical use of exosomes in oncology. The present invention describes the use of SELEX applied to intact tumor exosomes which allowed isolating new and unique aptameric molecules capable of specifically recognizing exosomes derived from BC and inhibiting the uptake thereof. Thus, the invention shows promising applications in both BC diagnosis and therapy.

BC is a leading cause of death in women and the early diagnosis thereof can greatly improve the success of therapies. Growing evidence shows that extracellular vesicles called exosomes can have an important potential as diagnostic and prognostic markers and as regulators of different neoplasms, including BC. Therefore, the discovery of molecules which recognize these vesicles with extreme selectivity is a major challenge.

Aptamers are small, single-stranded nucleic acids and represent ligands with high affinity and specificity for molecules of therapeutic interest.

The present invention describes the development of a new differential selection strategy (SELEX), called Exo-SELEX, to isolate aptamers capable of specifically recognizing exosomes derived from BC. Among the sequences identified with this approach, an aptamer (ex.50.T) capable of recognizing exosomes derived from blood samples of patients with BC was characterized and optimized. Furthermore, ex.50.T is shown to act as an inhibitor of the exosome uptake process and reduce in vitro exosome-induced migration. Therefore, this molecule provides an innovative tool for developing: non-invasive diagnostic methodologies based on the detection of tumor exosomes and new therapeutic approaches for breast cancer. The target of ex.50.T was also identified, i.e. the GREM1 protein, and it was observed that cells or exosomes overexpressing GREM1 cDNA increase their ability to be bound by ex.50.T. All these properties support the clinical applicability of this aptamer.

Preferably, the aptamer is a short RNA sequence (33 mer) containing 2′Fluoro-Pyrimidines which ensure good stability in human serum. Therefore, the molecule is well suited for use in diagnostic and in vivo tests. Future studies aimed at setting up diagnostic and therapeutic platforms based on aptamer-mediated targeting of exosomes are planned.

The molecule developed shows promising prospects for the realization of non-invasive methods for early BC diagnosis in liquid biopsy. Furthermore, the aptamer offers the possibility to be developed in innovative therapeutic strategies based on the specific removal of tumor exosomes. The development of specific aptamers can offer a highly innovative tool in both diagnosis and therapy which is comparable to monoclonal antibodies with the possibility of improving targeting efficiency associated with reducing production costs for clinical use. To date, there is great interest within the scientific community for aptamers and for the new and promising therapeutic opportunities which may result therefrom.

The molecule generated shows potential advantages in both diagnostic and therapeutic applications in BC. The high binding capacity of aptamers makes these molecules very promising diagnostic tools. In fact, the aptamers reveal good cost-effectiveness, high stability and can be easily modified for use within easy-to-use biosensors having a good detection limit (Ciancio D et al. Pharmaceuticals 2018). Ex-50 can be applied to the development of early and non-invasive diagnostic methodologies based on exosome detection for BC. This will offer a great advantage over conventional diagnostic strategies which primarily use invasive procedures, often not accessible to large populations and not applicable to repeated screening (Park, S M et al. Nat. Rev. 2017; Jafari S H J Cell Physiol. 2018). From a therapeutic point of view, the ex-50.T aptamer is capable of inhibiting the uptake of exosomes, showing potential interest in blocking tumor spread and cell transformation. The sequence developed shows important features, such as stability and no bind with serum albumin, which improve the potential applicability thereof. Furthermore, by virtue of the nature thereof, the aptamer could easily be further modified and optimized for use in vivo.

The selection approach developed is highly innovative and can be applied to different tumor systems to generate new molecules for diagnosis and therapy.

To our knowledge, there are no limits to the marketing of nucleic acid molecules for diagnostic and therapeutic purposes.

Therefore, an object of the present invention is a nucleotide aptamer or a variant thereof, where said aptamer comprises or essentially has the sequence:

(SEQ ID No. 1)
5′ GGGAAGAGAAGGACAUAUGAUCGUGAUAGCUGUGGCAGUUAAGAAU
AGAUCUUCGCUGCGAUUGACUAGUACAUGACCACUUGA 3′
in which the underlined sequence
(SEQ ID No. 2)
CGUGAUAGCUGUGGCAGUUAAGAAUAGAUCUUCGCUGCGA
can be replaced with
(SEQ ID No. 3)
UGGCGGCUCGUUGAAGGUCGUCUCACGGGCUAGGAAUGCG
or with
(SEQ ID No. 4)
ACGGCUGUUGCUUCGAAUGAACCAAGGUUCCUCGGCGCAA
or
a functional fragment thereof in which said
fragment comprises the sequence
(SEQ ID No. 2)
CGUGAUAGCUGUGGCAGUUAAGAAUAGAUCUUCGCUGCGA
or
(SEQ ID No. 3)
UGGCGGCUCGUUGAAGGUCGUCUCACGGGCUAGGAAUGCG
or
(SEQ ID No. 4)
ACGGCUGUUGCUUCGAAUGAACCAAGGUUCCUCGGCGCAA.

In an embodiment, the nucleotide aptamer or a variant thereof consists of the sequence:

(SEQ ID No. 1)
5′ GGGAAGAGAAGGACAUAUGAUCGUGAUAGCUGUGGCAGUUAAGAAU
AGAUCUUCGCUGCGAUUGACUAGUACAUGACCACUUGA 3′
in which the underlined sequence
(SEQ ID No. 2)
CGUGAUAGCUGUGGCAGUUAAGAAUAGAUCUUCGCUGCGA
can be replaced with
(SEQ ID No. 3)
UGGCGGCUCGUUGAAGGUCGUCUCACGGGCUAGGAAUGCG
or with
(SEQ ID No. 4)
ACGGCUGUUGCUUCGAAUGAACCAAGGUUCCUCGGCGCAA
or
a functional fragment thereof in which said
fragment comprises the sequence
(SEQ ID No. 2)
CGUGAUAGCUGUGGCAGUUAAGAAUAGAUCUUCGCUGCGA
or
(SEQ ID No. 3)
UGGCGGCUCGUUGAAGGUCGUCUCACGGGCUAGGAAUGCG
or
(SEQ ID No. 4)
ACGGCUGUUGCUUCGAAUGAACCAAGGUUCCUCGGCGCAA.
Preferably the aptamer of the invention comprises
or consists of the sequence
(SEQ ID No. 9)
5′ UGUGGCAGUUAAGAAUAGAUCUUCGCUGCGAUU 3′.

Preferably the pyrimidine residues of the aptamer or the variant thereof or a functional fragment thereof are replaced with 2′-fluoropyrimidine or said aptamer or the variant thereof or a functional fragment thereof is conjugated to at least one anticancer agent.

Preferably the aptamer or the variant thereof or a functional fragment thereof is for medical use, preferably for use in the treatment and/or prevention of a tumor or for use in the diagnosis of a tumor, preferably said tumor is a solid or hematological tumor.

The present invention also provides a method for selecting a nucleotide aptamer or a variant thereof comprising the steps of:

a) incubating a library of synthetic nucleotide oligomers with exosomes isolated from control cells, allowing the oligomers to bind thereto;

b) recovering a first series of nucleotide oligomers not bound to the exosomes isolated from control cells;

c) incubating said first series of nucleotide oligomers with exosomes isolated from target cells, allowing the first series of nucleotide oligomers to bind thereto;

d) recovering a second series of nucleotide oligomers bound to exosomes isolated from target cells;

e) amplifying said second series of nucleotide oligomers;

f) repeating steps a), b), c) and d) at least once and

g) selecting and sequencing nucleotide oligomers bound to exosomes isolated from target cells; wherein said nucleotide aptamer or a variant thereof binds specifically to exosomes isolated from target cells.

Preferably the synthetic nucleotide oligomers are labelled.

Preferably the nucleotide oligomers are oligoribonucleotides or modified nuclease-resistant oligoribonucleotides.

Preferably, the library used in step a) is a complex library of synthetic nucleotide oligomers. For complex library it is preferably intended a library comprising at least 10¹⁴ oligonucleotides, for example from 10¹⁴ to 10¹⁶ oligonucleotides.

Preferably, the library used in step a) is a RNA library prepared from the corresponding DNA library. Preferably, it comprises a pool of RNAs modified with 2′Fluoro-pyrimidines (2′F-Py).

Preferably, in step a) oligomers bind to exosomes in a short time frame, wherein for short time frame it is preferably intended a time comprised between 5 and 30 minutes.

Preferably the exosomes are conjugated to magnetic beads.

Preferably, the exosomes are whole exosomes.

Preferably the target cell is a tumor cell, preferably a breast cancer cell.

Preferably, said selected nucleotide aptamer or a variant thereof binds specifically to surface antigens of said exosomes isolated from target cells. The method indeed allows advantageously to select aptamers able of interfering with the recognition of exosomes toward the target cell with consequent inhibition of their uptake and of their protumorigenic effects.

The invention also provides a nucleotide aptamer or a variant thereof obtainable by the method described above.

The invention provides a pharmaceutical composition comprising the aptamer or the variant thereof as defined above, said composition preferably further comprising another therapeutic agent.

The invention provides a method for diagnosing a tumor in a patient from whom a sample was obtained, comprising:
incubating the sample with the aptamer or the variant thereof as defined above;
measuring the binding of the aptamer to the sample,

preferably said sample being a blood, serum or saliva sample, a biopsy, urine or cerebrospinal fluid.

The invention provides a kit for diagnosing a tumor comprising a nucleotide aptamer or the variant thereof as defined above.

The invention provides a nucleic acid coding for the aptamer or the variant thereof as defined above.

In the context of the present invention, the term “variant” also refers to longer or shorter polynucleotide sequences and/or polynucleotides having, for example, an identity percentage of at least 41%, 50%, 60%, 65%, 70% or 75%, more preferably of at least 85%, for example of at least 90%, and more preferably of at least 95% or 100% with the sequences described herein or with the complementary sequence thereof or with the corresponding DNA or RNA sequence thereof.

The term “variant” and the term “polynucleotide” also comprise modified synthetic oligonucleotides. These modified synthetic oligonucleotides are preferably locked nucleic acids (LNA), phosphoro-thiolated oligo, or methylated oligo, morpholines, 2′-O-methyl or 2′-O-methoxyethyl oligonucleotides and cholesterol-conjugated 2′-O-methyl modified oligonucleotides (antagomirs).

The term “variant” can also include nucleotide analogs, i.e., a ribonucleotide or a natural deoxyribonucleotide substituted by a non-natural nucleotide, and nucleic acids which can be generated by mutating one or more nucleotides or amino acids in the sequences thereof, or in the precursor sequences or equivalent. The term “variant” also comprises at least one functional fragment of the polynucleotide.

In the context of the present invention, “functional” means that it maintains the functional features of the aptamers from which they derive, e.g., the ability to bind BC exosomes and/or block the internalization thereof in target cells.

The term “variant” as used herein refers to compounds which have a similar but not identical chemical formula and share the same or substantially similar function as the compound with the similar chemical formula. In some embodiments, the analog is a mutant, variant, or modified sequence with respect to the unmodified or wild-type sequence on which it relies. In some embodiments, the analog is a functional fragment of any one of the nucleic acid sequences described herein. In some embodiments, the analog is a salt of any one of the nucleic acid sequences described herein. In such embodiments, the analog can retain about 99%, 98%, 97%, 96%, 95%, 94%, 93%, 92%, 91%, 90%, 85%, 80%, 75%, 70% or less biological activity with respect to the wild or natural-type sequences on which it is based.

Preferably, the aptamer or the variant thereof according to the invention selectively binds BC exosomes. Preferably, the aptamer or the variant thereof according to the invention is capable of discriminating BC exosomes from those derived from normal cells or other types of cancer (GBM and NSCLC).

Preferably, the aptamer or the variant thereof according to the invention is an RNA aptamer.

In the present invention, pyrimidine residues can also be modified as 2′-O-alkyl nucleotides, or with the addition of a capping at the 3′ end and locked nucleic acids or as LNA modifications to significantly increase RNA stability.

Preferably, the aptamer or the variant thereof is conjugated to at least one anticancer agent, said agent preferably being a nucleotide agent, such as a microRNA and/or a microRNA inhibitor and/or a siRNA and/or an ASO, or a drug or small molecule or therapeutic agent. The aptamer or the variant thereof can be conjugated to one or more of the above-mentioned agents, in any combination.

The high flexibility of the molecule also makes it suitable for further chemical modifications aimed at improving the pharmacodynamic and pharmacokinetic properties thereof, as well as for the use thereof in molecular imaging techniques. Possible chemical modifications are sugar, phosphate or nitrogen base substitutions and include: biotinylation, addition of fluorophores, incorporation of modified nucleotides, modification of pyrimidines in the 2′ position, addition of a capping at the 3′ end of the sequence, addition of a linker, conjugation with radioisotopes, contrast agents, chemical compounds, drugs or other therapeutic oligonucleotides.

A possible advantage of the invention is the opportunity to use the aptamer for the specific detection of BC tumor exosomes and the inhibition of the uptake thereof in target cells and the resulting pro-tumoral effects. In this perspective, the use of aptamers is a simple and ideal solution for developing biosensors and/or devices for the selective removal of circulating tumor exosomes. Indeed, the aptamer selectively binds epitopes of BC tumor exosomes.

The sequences of nucleic acids, protein or other agents of the present description can be administered, inter alia, as pharmaceutically acceptable salts, esters or amides. The term “salts” refers to inorganic and organic salts of the compounds of the present description. The salts can be prepared in situ during the final isolation and purification of a compound, or by separately reacting a purified compound in the free base or acid form thereof with a suitable organic or inorganic base or acid and isolating the salt thus formed. The representative salts include hydrobromide, hydrochloride, sulfate, bisulfate, nitrate, acetate, oxalate, palmitate, stearate, laurate, borate, benzoate, lactate, phosphate, tosylate, citrate, maleate, fumarate, succinate, tartrate, naphthylate, mesuco, gluco, lactobionate and lauryl sulfonate salts, and the like. The salts can include cations based on alkali and alkali earth metals, such as sodium, lithium, potassium, calcium, magnesium, and the like, as well as non-toxic ammonium, quaternary ammonium, and amine cations including, but not limited to, ammonium, tetramethylammonium, tetraethylammonium, methylamine, dimethylamine, trimethylamine, triethylamine, ethylamine, and the like; see S. M. Berge, et al., J Pharm Sci, 66: 1-19 (1977), describing the saline forms of nucleic acids and incorporated by reference in the entirety thereof.

Typically, an effective amount of the compounds of the invention, sufficient to obtain a therapeutic or prophylactic effect, ranges from about 0.01 mg per kilogram of body weight per day to about 100 mg per kilogram of body weight per day. Preferably, the dosage ranges from about 0.1 mg per kilogram of body weight per day to about 50 mg per kilogram of body weight per day. Preferably, the dosage ranges from about 0.5 mg per kilogram of body weight per day to about 10 mg per kilogram of body weight per day. A therapeutically effective amount of a pharmaceutical composition comprising one or more of any of the nucleic acid sequences described herein can also be administered in combination with two, three, four or more nucleic acid sequences described herein, or with one or more additional therapies.

Another object of invention is a pharmaceutical composition comprising the aptamer or the variant thereof as defined above, said composition preferably further comprising another therapeutic agent. Examples of further therapeutic agents are conventional chemotherapeutic drugs such as anthracyclines (epirubicin and doxorubicin), taxanes (docetaxel and paclitaxel), fluorine derivatives (5-fluorouracil and capecitabine), platinum derivatives (e.g., cisplatin and carboplatin); molecular target drugs such as monoclonal antibodies (i.e., trastuzumab, and bevacizumab); small molecules having direct or indirect immune-mediated anticancer activity.

Another object of the invention is a nucleic acid coding for the aptamer or the variant thereof as defined above, a vector comprising said nucleic acid and a cell comprising said nucleic acid or said vector. The aptamer according to the invention is preferably nuclease resistant.

A further object of the invention is the aptamer or the variant thereof according to the invention for use as a carrier of molecules inside cancer cells.

The conjugate according to the invention can comprise at least one aptamer as described above and at least one anticancer agent as defined above.

The present invention will be illustrated with the aid of non-limiting examples referring to the following figures and tables.

TABLE 1
SELEX cycle conditions
Table 1. SELEX conditions.
The amount of RNA, incubation time, number of washes after positive selection,
cell lines used as a source of exosomes and the presence of competitor during the
different cycles of Exo-SELEX are indicated. Comp.: competitor (t-RNA, 0.1 mg/mL).

| | RNA | Time | | Counter- | Positive | |
|---|---|---|---|---|---|---|
| # | (pmoles) | (min) | Washes | selection | selection | Comp. |
| 1 | 800 | 30 | 1 | Pt. 100; Pt. 101 | Pt. 46 (HER⁺) | NO |
| 2 | 800 | 30 | 2 | Pt. 100; Pt. 101 | Pt. 46 (HER⁺) | NO |
| 3 | 600 | 30 | 3 | Pt. 72 | Pt. 46 (HER⁺); | NO |
| | | | | | Pt. 170 (TNBC) | |
| 4 | 800 | 30 | 4 | Pt. 72; Pt. 44 | Pt. 46 (HER⁺); | NO |
| | | | | | Pt. 37 (HER⁺) | |
| 5 | 800 | 30 | 5 | Pt. 72; | Pt. 46 (HER⁺); | NO |
| | | | | Pt. 100; Pt. 101 | Pt. 170 (TNBC) | |
| 6 | 800 | 30 | 5 | Pt. 72; Pt. 44 | Pt. 46 (HER⁺); | Yes |
| | | | | | Pt. 37 (HER⁺) | |
| 7 | 800 | 30 | 5 | Pt. 72; Pt. 44 | Pt. 46 (HER⁺); | Yes |
| | | | | | Pt. 37 (HER⁺) | |
| 8 | 800 | 30 | 5 | Pt. 72; Pt. 44 | Pt. 46 (HER⁺); | Yes |
| | | | | | Pt. 37 (HER⁺) | |

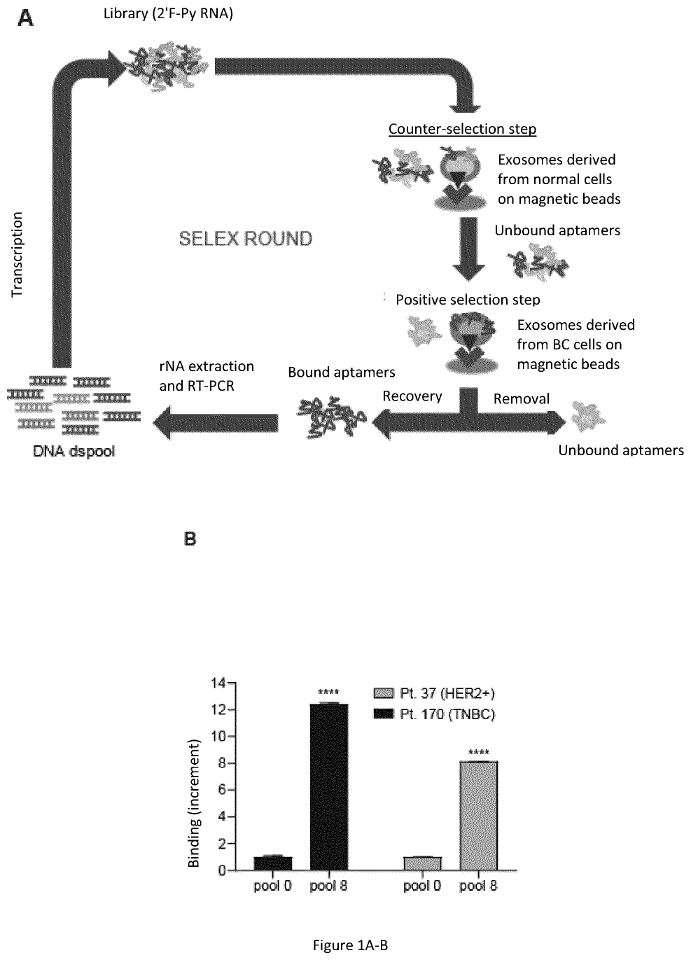

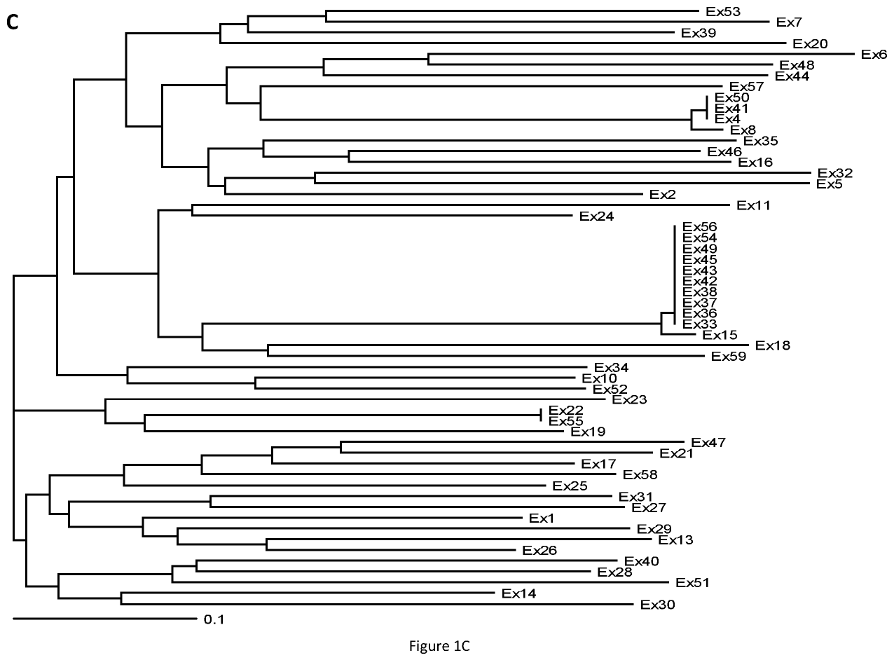

FIG. 1. A) exo-SELEX diagram. B) Binding of the initial and final library of exo-SELEX analyzed by means of RT-qPCR.

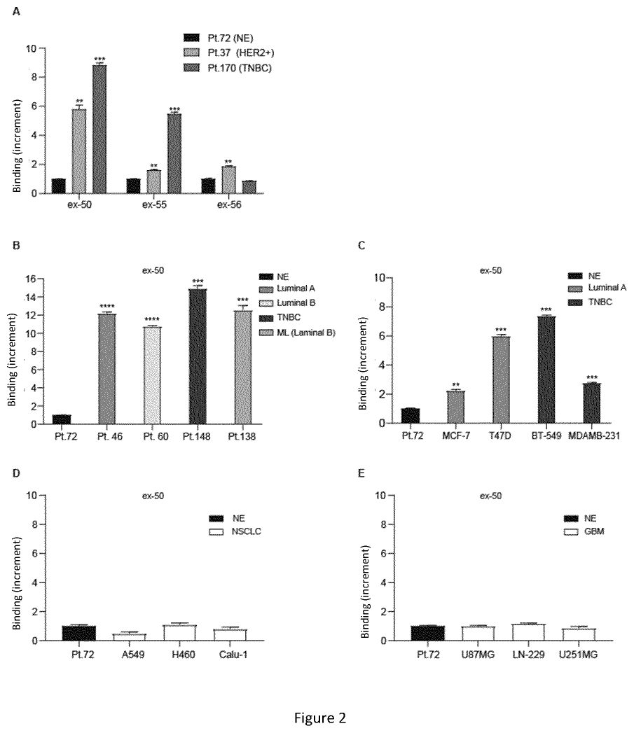

FIG. 2. Binding analysis of individual sequences. A) Binding capacity of the three most enriched sequences (ex-50, ex-55, ex-56) on exosomes derived from normal (Pt.72, NE) or cancerous (Pt.37 and Pt.170) primary breast cells analyzed by means of RT-qPCR. B) Analysis by means of RT-qPCR of the binding of ex-50 on exosomes derived from Pt.72 (NE), different primary BC cultures or from cells of a luminal B BC metastatic lymph node (MHL). C-E) Binding by means of RT-qPCR of ex-50 on exosomes derived from the continuous cell lines indicated. In (A-E) the results are expressed as increase with respect to binding on exosomes derived from the NE control cells. The error bars show the SD values. **, p<0.01; ***, p<0,001; ****, p<0,0001.

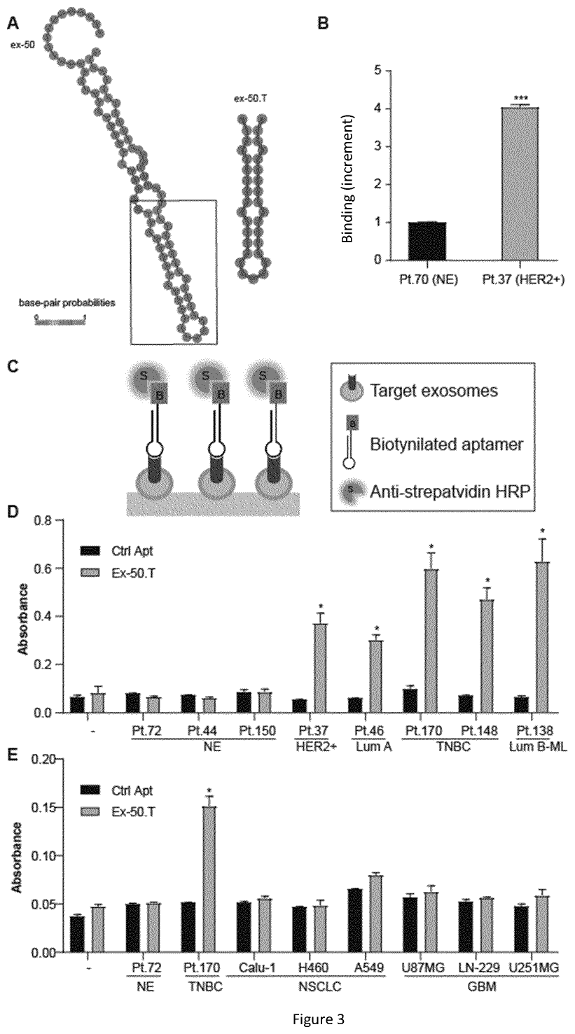

FIG. 3. Ex 50.T binding. A) Diagram of the secondary structure of ex-50 and the derived short sequence (ex-50.T). B) Ex 50.T binding capacity analyzed by means of RT-qPCR on exosomes derived from normal (Pt.72, NE) or cancerous (Pt.37) primary breast cells. C) Diagram of the ELONA assay used to measure aptamer binding to exosomes. C, D) Binding assays for ELONA to measure ex-50.T binding or binding of a control aptamer (Ctrl Apt) on exosomes derived from: several primary lines (Pt.) of normal epithelial cells (NE) or BC cells (HER2 positive: HER2+; Luminal A: Lum A; Triple-negative BC: TNBC), from a BC luminal B metastatic lymph node (Lum B-ML), or from several continuous lines of GBM or NSCLC. *, p<0.05 (t-test versus NE).

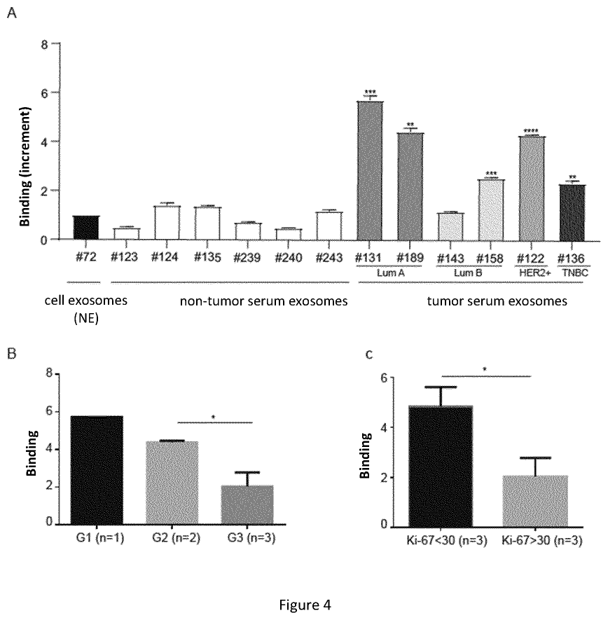

FIG. 4. Binding of ex-50.T on exosomes derived from liquid biopsies. A) Assay of ex-50 T binding by means of RTqPCR on serum-derived exosomes from 6 patients with BC and 6 non-cancer patients. B) Correlation between tumor degree of BC patients tested and ex-50 T binding intensity. C) Correlation between the Ki-67 level of patients with BC (Ki-67<30 and Ki-67>30) and the binding of ex-50.T. NE, normal epithelial; Lum, luminal. Statistics for test t: *, p<0.05. **, p<0.01; ***, p<0.001; ****, p<0.0001.

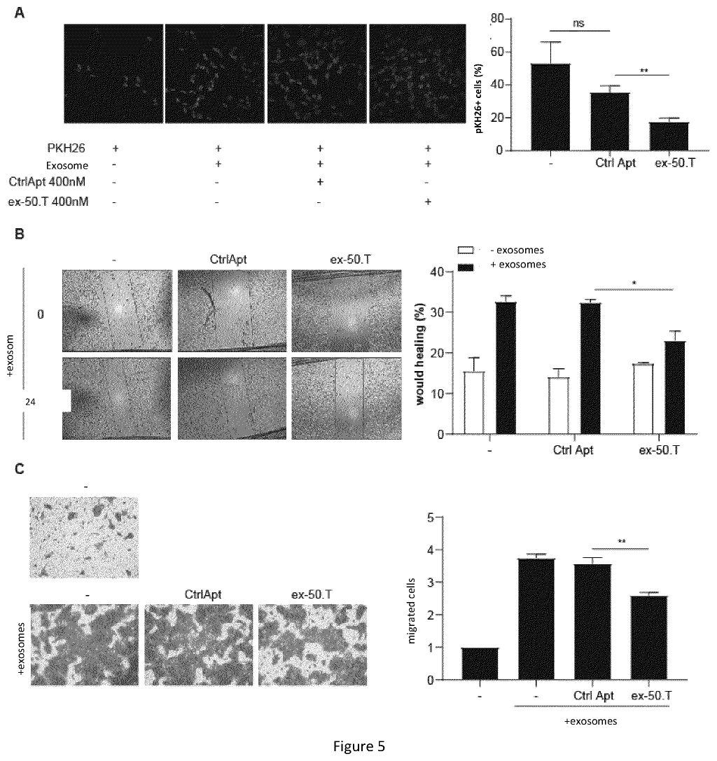

FIG. 5. Functional activity of ex-50.T. A) The ability of ex-50.T to inhibit the uptake of exosomes derived from TNBC MDA-MB-231 cells was assessed by incubating cells with tumor exosomes labeled with PKH26 and treated or untreated with ex-50.T or the control sequence (CtrlApt). B) The ability of ex-50.T to inhibit the pro-migratory effect of MCF-7 BC cells induced by exosomes derived from TNBC MDA-MB-231 cells was measured by means of wound healing assay in the presence of tumor exosomes treated or not treated with ex50.T or CtrlApt. C) The ability of ex-50.T to inhibit the pro-migratory effect of normal MCF-10A breast cells induced by exosomes derived from TNBC MDA-MB-231 cells was measured by means of boyden-chamber assay in the presence of tumor exosomes treated or not treated with ex50.T or CtrlApt.

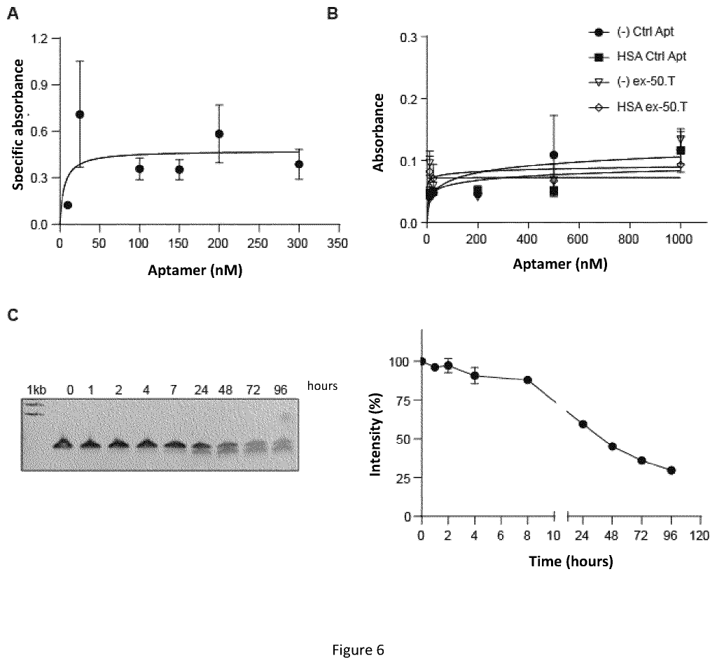

FIG. 6. A) Affinity of ex-50.T on MDA-MB-231-derived exosomes by means of ELONA. The specific binding of ex-50.T was measured by subtracting the values obtained with the CtrlApt. Kd estimated around 6 nM. B) Binding of ex-50.T to bovine serum albumin by means of ELONA. C) Stability in 85% serum of ex-50.T.

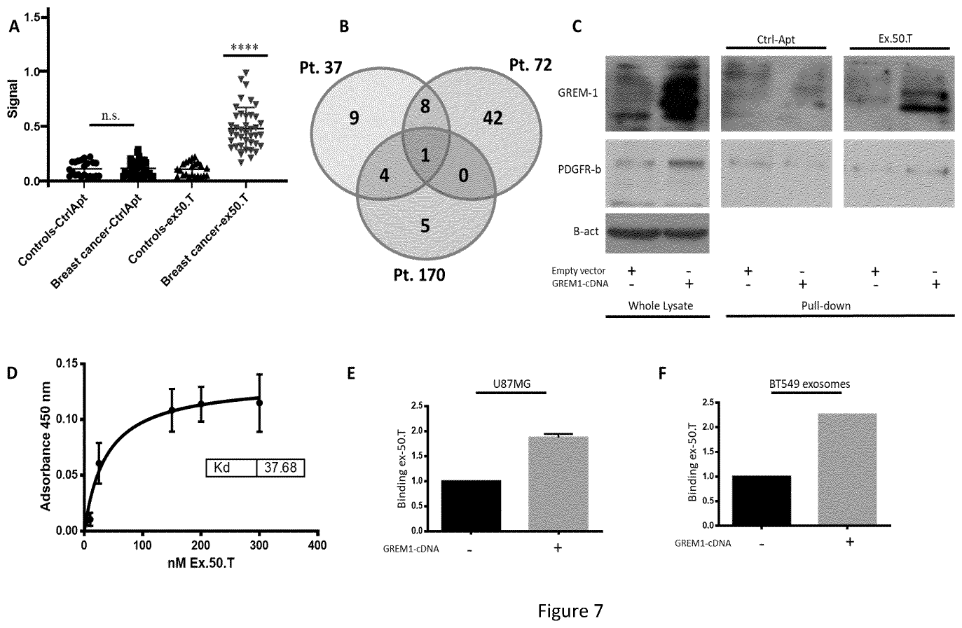

FIG. 7. (A) ElONA assay on exosome derived from breast cancer patients and control patients. (B) Venn Diagram of proteomic analysis of proteins of ex.50.T pull down experiments on exosome derived of Pt. 72 (fibroadenoma) Pt. 37 and 170. Protein from scrambled sequences were subtracted as background. (C) Western Blot analysis of whole cell lysate of U87MG upon transfection with GREM1 encoding vector confirming GREM1 as target of ex.50.T. (D) ELONA is used to prove ex50.T binding to recombinant human GREM1 and to calculate Kd of the ex50.T-GREM1 complex (E) Binding assay of ex.50.T on U87MG cells upon transfection of a vector encoding GREM1. (F) Binding assay of ex.50.T on BT459 derived exosomes upon transfection of a vector encoding GREM1 in parental cells.

### DETAILED DESCRIPTION OF THE INVENTION

### Examples

Materials and Methods

Commercial cell lines, cell lines derived from patient samples, and patient blood were used. The patients involved in the study were informed and we requested informed consent. The ethics committee of the University of Naples approved the recruitment and sampling protocols (study no. 119/15ES1).

Cell Cultures and Exosome Isolation

Primary cultures of primary BC or normal epithelial cells (derived from normal breast hyperplasia) were prepared from surgical samples of human biopsies (provided by Clinica Mediterranea S.p.a) as previously reported (Donnarumma E, et al. Oncotarget. 2017). The cells were maintained in DMEM/Nutrient F12-Ham growth medium (DMEM-F12) (Sigma-Aldrich).

The continuous cell lines were purchased from ATCC (standard LG, Milan, Italy) and grown in the following mediums: BC cells T47D, BT549 and MDA-MB-231, NSCLC cells A549 and H460: Roswell Park Memorial Institute (RPMI); BC MCF-7 cells, NSCLC Calu-1 cells, GBM U87MG and U251MG cells: DMEM; LN-229 GBM cells: DMEM-advanced; MCF10A cells: DMEM/F12 supplemented with EGF (20 ng/ml), hydrocortisone (0.5 μg/ml), cholera toxin (100 ng/ml), insulin (10 μg/ml), horse serum (5%). All the mediums were supplemented with 10% fetal bovine serum (FBS, Sigma-Aldrich.

For the exosome preparation, the cells were grown in medium with 10% FBS-Exo⁻ (exosome depleted, SBI) in 150 mm plates (15 ml volume). The exosomes were isolated from the growth medium using the ExoQuick-TC™ kit (SBI) according to the protocol in the instructions.

RNA Preparation and In Vitro Transcription

For the SELEX procedure, we used a 2′-F-Py modified RNA library which was prepared from the corresponding DNA library (from TriLink Biotech) with a degenerated central region of 40 nucleotides flanked by two constant sequences required for amplification and transcription. The library or single long sequences were amplified by means of PCR using the following primers: forward (with T7-RNA polymerase promoter): 5′-TTCAGGTAATACGACTCACTATAGGGAAGAGAAGGACATATGAT-3′ (SEQ ID No. 5); reverse: 5′-TCAAGTGGTCATGTACTAGTCAA-3′ (SEQ ID No. 6). The program used was: 10 cycles of: 30 seconds at 95° C., 30 seconds at 64° C. and 1 minute at 72° C.

The in vitro transcription of the library or individual sequences was performed in the presence of 1 mM 2′-F-Py using a mutant form of T7 RNA polymerase (Epicentre Biotechnologies). The DNA template was incubated at 37° C. o.n. in a transcription mixture containing: 1× transcription buffer, 1 mM 2′F-Py (2′F-2′-dCTP and 2′F-2′-dUTP, TriLink Biotech, San Diego, Calif.), 1 mM ATP, 1 mM GTP (Thermo scientific), 10 mM dithiothreitol (Thermo scientific), 0.5 u μl RNAse inhibitors (Roche), 5 μg/ml inorganic pyrophosphatase (Roche) and 1.5 u/μl T7-RNA polymerase. After transcription, the remaining DNA was removed by means of DNase I digestion (Roche) and the resulting sequences were recovered with phenol:chloroform extraction and ethanol precipitation. The RNAs were purified over 8% acrylamide/7 M urea denaturing gel.

The short aptamers were purchased from Tebu-bio srl.

Exosome-SELEX

For the SELEX strategy, the exosomes were isolated from the cell growth medium (15 ml medium), quantified by means of Bradford (Bio-rad) and 40 μg were conjugated with magnetic beads containing anti-CD81 antibodies (ThermoFisher Scientific) at 4° C. o.n. Prior to each SELEX cycle, the pool of 2′F-Py RNA was diluted in PBS and subjected to denaturation/renaturation steps of 85° C. for 5 minutes, ice for 2 minutes, 37° C. for 10 minutes. The library was first incubated for 30 minutes at room temperature with exosomes derived from normal cells (NE) bound to the magnetic beads with gentle rotation (counter-selection step). The unassociated RNAs were recovered using a magnetic separator (Millipore) and incubated with exosomes derived from cancer cells conjugated with the beads for 30 minutes with gentle rotation (selection step). The aptamer-exosome complexes were recovered with the magnetic separator, washed with PBS and suspended in euroGOLD TriFast (Euroclone) for the RNA extraction. The recovered RNA was amplified by means of RT-PCR and transcribed for the following SELEX cycle (see Table 1 for the conditions). When exosomes from multiple lines were included in the cycle, an equal amount of exosomes (for a total of 40 μg) from each sample was pooled and used as a target.

The reverse-transcription was performed using the enzyme M-MuLV (Roche). The product obtained was then amplified by means of error-prone PCR in the presence of high concentrations of MgCl2 (7.5 mM) and dNTP (1 mM) in order to introduce random mutations. The PCR program used was: 30 seconds at 95° C., 1 minute at 64° C. and 30 seconds at 72° C.

Isolation of Individual Aptamers

The individual sequences were obtained by means of cloning the final pool with the kit TOPO-TA (Invitrogen), according to the manufacturer's protocol. The transformation was performed with Escherichia coli DH5α (Invitrogen). Individual white clones were recovered, the DNA was extracted with the MINIPREP kit (Qiagen) and sequenced by Eurofins Genomics by means of T7 primer. To derive the shortened sequence, the secondary structure of the aptamer was analyzed with RNAFold (http://rna.tbi.univie.ac.at).

The sequences of long aptamers are:

Ex-50:
(SEQ ID No. 1)
5′ GGGAAGAGAAGGACAUAUGAUCGUGAUAGCUGUGGCAGUUAAGAAU
AGAUCUUCGCUGCGAUUGACUAGUACAUGACCACUUGA 3′
Ex-55:
(SEQ ID No. 7)
5′ GGGAAGAGAAGGACAUAUGAUUGGCGGCUCGUUGAAGGUCGUCUCA
CGGGCUAGGAAUGCGUUGACUAGUACAUGACCACUUGA 3′
Ex-56:
(SEQ ID No. 8)
5′ GGGAAGAGAAGGACAUAUGAUACGGCUGUUGCUUCGAAUGAACCAA
GGUUCCUCGGCGCAA UUGACUAGUACAUGACCACUUGA 3′

The central variable region is underlined.

The sequence of ex-50.T is:

(SEQ ID No. 9)
5′ UGUGGCAGUUAAGAAUAGAUCUUCGCUGCGAUU 3′

Binding Test by Means of RT-qPCR

For the binding assays, the RNA sequences were diluted in PBS, subjected to denaturation/renaturation steps and incubated at a concentration of 200 nM with exosomes bound (13 μg) to magnetic beads for 30 minutes with slight rotation at room temperature. The unassociated RNAs were removed with the magnetic separator by washing 4 times with PBS. The associated RNAs were recovered from TRIzole containing 0.5 pmol/mL of an unrelated aptamer (CL4:

(SEQ ID No. 10)
5′-GCCUUAGUAACGUGCUUUGAUGUCGAUUCGACAGGAGGC-3′

used as an internal reference and amplified. Briefly, the recovered RNAs were reverse-transcribed using the reverse primer with the following protocol: 65° C. for 5 minutes, 22° C. for 5 minutes, 42° C. for 30 minutes, 48° C. for 30 minutes, 95° C. for 5 minutes. The RT reaction products were amplified with iQ SYBR Green Supermix (Bio-Rad, Hercules, Calif.) with the following protocol: initial heating step at 95° C. for 2 minutes, followed by 40 cycles of 95° C. for 30 s, 55° C. for 30 s, 60° C. for 30 s. The amounts of RNA recovered were calculated in relation to a standard curve of known RNA concentrations and normalized with respect to the internal reference CL4.

ELONA-Based Test

For ELONA, wells of a 96-well High Binding microplate (Nunc MaxiSorp) were left uncoated or coated with exosomes (4 μg) o.n. All the subsequent steps were performed at room temperature. Following incubation, the plate was blocked with 300 μl 3% BSA (AppliChem) in PBS for 2 hours and incubated with 100 μL biotinylated aptamers (Tebu-bio), dissolved in PBS for 2 hours. Then the plate was washed three times with 300 μl PBS and streptavidin-HPR dissolved in 100 μl PBS (Sigma Aldrich) was added with dilution 1:10000. After 1 hour of incubation, the plate was washed three times before the addition of the TMB developing solution. The reaction was stopped with 0.16 M sulfuric acid and signal strength was analyzed by measuring absorbance at 450 nm with a microplate reader (Thermo Scientific).

MDA-MB-231-derived exosomes and increasing concentrations of aptamer (ex-50.T or control) were used to calculate the binding affinity. The Kd was determined by means of GraphPad Prism. To verify the binding on human serum alumina (HSA), the plate coating was performed at 30 nM HSA (Sigma Aldrich).

Serum Sample Exosome Binding Assay

On the day patients underwent surgery to remove the tumor mass, serum samples (yellow cap Vacutainer) were collected and subsequently stored at −80° C. The exosomes were extracted with the Norgen Biotek Corporation serum/plasma isolation kit. 13 μg of exosomes were conjugated to anti-CD81 magnetic beads o.n. at 4° C. The binding was measured by means of RT-qPCR as described above. Six normal serum samples and six serum samples from patients with BC were selected. No age difference was found in the group (mean normal vs. tumor SD, 43.8±5.26 vs. 50.5±7.31).

Serum Stability

To verify the serum stability, that aptamer was incubated at a concentration of 4 μM in 85% human serum (from (human serum type AB, Euroclone) from 1 to 96 hours. At each time interval, the RNA (16 pmols) was recovered and incubated for 1 h at 37° C. with proteinase K in order to remove the serum proteins prior to electrophoretic migration in 15% denaturing polyacrylamide gel. The gel was stained with ethidium bromide and viewed by means of exposure to UV rays. The bands were quantified with the program Image J NIH.

Immunofluorescence

The MDA-MB-231 cells were cultured on glass slides in 24-well plates in exosome-free 10% FBS medium. The exosomes isolated from MDA-MB-231 were labeled with PKH26, a fluorescent cell membrane dye with red emission (Sigma-Aldrich). For the labeling, the exosomes were incubated for 5 min at room temperature with PKH26 (diluted 1:1000 in PBS) and then an equal volume of 1% BSA was added to stop the reaction. To recover the labeled exosomes, the samples were ultracentrifuged for 70 min at 100,000×g at 4° and washed with 4 ml of PBS. The pellet containing labeled exosomes was resuspended in FBS-free culture medium and quantified by means of Bradford (Bio-rad). The exosomes (40 μg for each test point) were incubated in the dark with denatured aptamers (400 nM) for 30 minutes with gentle rotation at room temperature. The cells were treated for 3 hours with exosomes pre-incubated or not with aptamers, washed three times with PBS, fixed with a mixture of Methanol/Acetone (1:1) for 10 minutes at −20° C. The slides were washed twice with PBS and mounted with Gold antifade reagent with DAPI (Invitrogen) and viewed by means of confocal microscopy.

Wound Healing Test

MCF-7 breast cancer cells (2×105) were plated in 24-well multiwells in DMEM with exosome-free 10% FBS. After 24 h, the cells were scraped to induce a wound, washed with PBS and treated with 50 μg MDA-MB-231-derived exosomes pre-incubated or not with aptamers in serum-free DMEM. Photos were taken immediately after wound creation and after 24 hours.

Migration Test by Means of Boyden Chamber

MCF10A cells (5×10⁴ cells) were plated in the upper chamber of a 24-well transwell (Corning Incorporate, Corning, N.Y.) in DMEM/F12 with exosome-free 0.5% FBS containing 20 μg MDA-MB-231-derived exosomes pre-incubated or not with the aptamer ex-50.T or CtrlApt (400 nM). The medium 10% FBS was used as chemo attractor. After 48 hours, the migrated cells were stained with 0.1% crystal violet in 25% methanol. The percentage of migrated cells was evaluated by means of the program Image J NIH.

Pulldown Experiments

Protein identification was performed with an adapted protocol described previously by Berezovski et al. (Berezovski M. V., Lechmann M., Musheev M. U., Mak T. W., Krylov S. N. Aptamer-facilitated biomarker discovery (AptaBiD) J. Am. Chem. Soc. 2008) and Affinito et al (Affinito A, Quintavalle C, Esposito C L, Targeting Ephrin Receptor Tyrosine Kinase A2 with a Selective Aptamer for Glioblastoma Stem Cells. Mol Ther Nucleic Acids. 2020 Jun. 5; 20:176-185. doi: 10.1016/j.omtn.2020.02.005. Epub 2020 Feb. 13. PMID: 32169805; PMCID: PMC7068199.). To isolate protein, cells or exosomes were lysed with sodium deoxycholate (Sigma-Aldrich) at 0.1% (w/v) in 10 mM PBS with Ca2+, Mg2+(Sigma-Aldrich) and incubated with a biotinylated-scrambled at 200 nM for 30 min at room temperature, as a counterselection step. Then, lysates were incubated with streptavidin MagneSphere paramagnetic particles (Promega, Milan, Italy) for 30 min at 4° C. Afterward, unbound proteins were incubated with biotinylated A40s (TriLinK Biotechnologies) at 200 nM for 30 min at room temperature. Ex.50.T-protein complexes were incubated with streptavidin MagneSphere paramagnetic particles (Promega) for 30 min at 4° C. Collected beads were washed twice with cold 10 mM PBS with Ca2+, Mg2+(Sigma-Aldrich). Thus, incubation with 8 M urea for 1 h at 4° C. led to protein denaturation and release from the aptamer and magnetic beads. Pull-down proteins were analysed by mass spectrometry or separated by SDS-PAGE (12% polyacrylamide gel), transferred to nitrocellulose membranes (Millipore, Bedford, Mass., USA), and immunoblotted for GREM1 antibody (Cell Signalling Technology).

Western Blot

Exosomes or cells were lysed in RIPA buffer (Thermo Scientific, Pierce RIPA buffer 89900) containing Protease Inhibitor (Roche, Protease inhibitor cocktail tablets 11836145001) and Phosphatases inhibitor (Sigma, Phosphatase inhibitor cocktail 3) for 2 hours in ice and the protein concentration was determined by the Bradford assay (Bio-Rad, Protein Assay Dye Reagent Concentrate 500-0006). Then, 16 μg of protein was separated by 10% SDS-PAGE and blotted onto a nitrocellulose membrane (GE Healthcare). The membranes were stained with a Ponceau S solution (Sigma) and then washed with Tris-buffered saline containing 0.1% Tween-20 (T-TBS). After blocking with 5% No Fat Dry Milk in T-TBS, the membranes were incubated at 4° C. overnight with the following primary antibodies: anti-b-Actin (Sigma Aldrich) and anti-PDGFR-b (Cell signalling) and anti GREM1 (Cell Signalling Technology). Detection was performed by peroxidase-conjugated secondary antibodies using the ultra-enhanced chemiluminescence system (Thermo Scientific).

KD Determination

The KD for the ex50.T-recombinant human GREM1 complex was determined by performing an aptamer-based ELONA, as previously described. Fitting curves were designed by using GraphPad Prism 6 software.

Statistical Analysis

The statistics were analysed with the t test for comparisons between two groups as indicated.

Results

In order to generate aptamers capable of selectively binding BC exosomes, a new and innovative differential SELEX procedure, referred to as exo-SELEX, was developed.

The procedure uses exosomes as selection targets. To have a simple manner to recover the bound aptamers, the whole exosomes were conjugated to magnetic beads. Such a technique therefore extends the use of magnetic beads hitherto applied to the immobilization of purified proteins, to the capture of whole exosomes while preserving the physiological structure thereof. The selection approach is able to combine the simplicity of SELEX on protein with the maintenance of the physiological state of the typical SELEX cell targets.

Specifically, exosomes derived from normal primary epithelial or tumor cells are used as targets for counter-selection and selection, respectively. A pool of RNAs modified with 2′Fluoro-pyrimidines (2′F-Py) was used as the starting library to increase nuclease resistance. At each exo-SELEX cycle (FIG. 1a), the library was first incubated on exosomes derived from normal epithelial primary cells and conjugated to magnetic beads (counter-selection). The unbound sequences were recovered and incubated with exosomes derived from primary BC cells and conjugated to beads (selection). The bound sequences were then recovered by means of RNA extraction and amplified for RT-PCR.

After performing 8 cycles of exo-SELEX (at least 8 cycles, at least 10 cycles), the final library effectively enriched with aptamers capable of binding BC exosomes (FIG. 1b) was cloned and a panel of 57 individual sequences were sequenced and grouped into families (FIG. 1c). Three distinct groups of sequences were identified with three more-enriched aptamers (ex-50, ex-55 and ex-56). The ability of these aptamers to discriminate normal exosomes with respect to those of breast cancer was then analyzed (FIG. 2a).

Among the sequences, the best binding properties were identified for the aptamer ex-50. The ability of ex-50 to effectively discriminate BC exosomes was confirmed by means of binding assays on exosomes derived from other BC patients (FIG. 2b) or on a panel of exosomes derived from continuous BC cell lines (FIG. 2c) or from different cancer types, NSCLC (FIG. 2d) and GBM (FIG. 2e).

The aptamer ex-50 was then optimized by deriving a short version (ex-50.T) of 33 mer (FIG. 3a) with the same binding properties. This molecule is capable of effectively and highly specifically discriminating exosomes derived from BC cells from those originating from normal epithelial cells and also from cells of different tumors such as GBM and non-small cell lung cancer (FIG. 3b-e). Fundamentally, the aptamer ex-50.T was capable of recognizing the BC exosomes derived from patient blood samples (FIG. 4a), showing a negative correlation of the bind with the degree of the tumor (FIG. 4b) and Ki-67 positive cell percentage (FIG. 4c). This data demonstrates the usefulness of ex-50.T as a tool for the early diagnosis of BC.

In addition to recognizing BC exosomes, ex-50.T is also endowed with an inhibitory action, being able to block the uptake thereof and inhibit exosome-dependent cell migration (FIG. 5).

Ex50.T showed excellent binding affinity for BC exosomes, good serum stability and non-binding to human serum albumin (FIG. 6).

Furthermore ex.50.T showed a good ability in an ELONA assay to discriminate exosomes derived from serum of breast cancer patients compared to exosome derived from serum of control patients (FIG. 7A). Pull down experiments followed by proteomic analysis revealed that 5 protein were in common between protein pulled down in BC exosomes and not in control exosomes (FIG. 7B). GREM-1 was one identified protein. Experiment of pull down on whole lysate of U87MG confirmed that GREM1 is the target of ex.50.T (FIG. 7C). Furthermore we observed that ex.50.T binds with good affinity to GREM1 with a kd of 37.68 nM (FIG. 7D) and cells or exosomes upon overexpression of GREM1 cDNA increase their ability to be bound by ex.50.T (FIG. 7E-F). All these properties increase the clinical applicability thereof.

The selection protocol described herein concerns the SELEX methodology applied to intact tumor exosomes. It offers a new, innovative and simple differential approach to isolate aptameric molecules specifically capable of recognizing exosomes derived from cells of interest. The strategy therefore shows a wide applicability to different types of tumors, as well as to different clinical problems, including cardiovascular diseases and neurological disorders, which may benefit from exosome targeting.

The molecule obtained represents a specific aptamer for BC exosomes. So far, protocols for detecting exosomes employing oligonucleotides against known targets (such as CD63 and EpCAM) have been described (Zhang K, et al. ACS Sens. 2019; Wang L et al. ACS Appl Mater Interfaces. 2020; Xie X et al Nat Commun. 2019). The use of the aptamer of the invention allows a highly specific (not currently present) recognition necessary for the clinical applicability of these detection systems.

The aptamer of the invention is an example of an aptamer against tumor exosomes having inhibitory function and capable of blocking the uptake of BC exosomes. Recently, exosomes have been shown to be important mediators of communication between tumor cells and the stroma, playing a key role in tumor progression. Therefore, the inhibition of tumor exosome uptake by surrounding tissues is an effective strategy for interfering with tumor spread and development (Sun W et al. Acta Pharmacol Sin. 2018). By virtue of the functional properties of ex-50 T, it represents a useful molecule for blocking tumor spread and cell transformation.

Therefore, ex50.T shows high potential for the development of new non-invasive methods for the early detection of BC and innovative personalized therapeutic approaches for this tumor. The high flexibility of the molecule also makes it suitable for further chemical modifications aimed at improving the pharmacodynamic and pharmacokinetic properties thereof, as well as for the use thereof in molecular diagnosis techniques.

### BIBLIOGRAPHIC REFERENCES

1. Harbeck N, Gnant M. Breast cancer. Lancet. 2017; 18(389): 1134-50.
2. Rugo H S, Olopade O I, DeMichele A, Yau C, van't Veer L J, Buxton M B, et al. Adaptive randomization of veliparib-carboplatin treatment in breast cancer. N Engl J Med. 2016; 375(1): 23-34.
3. Joyce D P, Kerin M J, Dwyer R M. Exosome-encapsulated microRNAs as circulating biomarkers for breast cancer. Int J Cancer. 2016; 139(7): 1443-8.
4. Hannafon B N, Ding W Q. Intercellular communication by exosome-derived microRNAs in cancer. Int J Mol Sci. 2013; 14(7): 14240-69.
5. Lowry M C, Gallagher W M, O'Driscoll L. The role of exosomes in breast cancer. Clin Chem. 2015; 61(12): 1457-65.
6. Chia B S, Low Y P, Wang Q, Li P, Gaoa Z. Advances in exosome quantification techniques Trends Anal. Chem. 2017; 86: 93-106.
7. Rashed M H, Bayraktar E, Helal G K, Abd-Ellah M F, Amero P, Chavez-Reyes A, et al.

Exosomes: From Garbage Bins to Promising Therapeutic Targets Int J Mol Sci. 2017; 18(3): 538.
8. Hoshino A, Costa-Silva B, Shen T L, Rodrigues G, Hashimoto A, Tesic Mark M, et al. Tumour exosome integrins determine organotropic metastasis. Nature. 2015; 527(7578): 329-35.
9. Donnarumma E, Fiore D, Nappa M, Roscigno G, Adamo A, Iaboni M, et al. Cancer-associated fibroblasts release exosomal microRNAs that dictate an aggressive phenotype in breast cancer. Oncotarget. 2017; 8(12): 19592-608.
10. Bliss S A, Sinha G, Sandiford O A, Williams L M, Engelberth D J, Guiro K, et al.

Mesenchymal stem cell-derived exosomes stimulate cycling quiescence and early breast cancer dormancy in bone marrow. Can Res. 2016; 76(19): 5832-44.
11. Huang M B, Gonzalez R R, Lillard J, Bond V C. Secretion modification region derived peptide blocks exosome release and mediates cell cycle arrest in breast cancer cells. Oncotarget. 2017; 8(7): 11302-15.
12. Taylor D D, Gercel-Taylor C. Exosomes/microvesicles: mediators of cancer associated immunosuppressive microenvironments. Semin. Immunopathol. 2011; 33: 441-54.
13. Wang M, Ji S, Shao G, Zhang J, Zhao K, Wang Z, et al. Effect of exosome biomarkers for diagnosis and prognosis of breast cancer patients Clin Transl Oncol. 2018; 20(7): 906-11.
14. Cheng N, Du D, Wang X, Liu D, Xu W, Luo Y, et al. Recent Advances in Biosensors for Detecting Cancer-Derived Exosomes. Trends in Biotechnology 2019; 37(11): 1236-54.
15. Wu X, Chen J, Wu M, Zhao J X. Aptamers: Active Targeting Ligands for Cancer Diagnosis and Therapy. Theranostics 2015; 5(4): 322-44.
16. Catuogno S, Esposito C L. Aptamer Cell-Based Selection: Overview and Advances. Biomedicines. 2017; 5(3): E49.
17. Maimaitiyiming Y, Hong F, Yang C, Naranmandura H. Novel insights into the role of aptamers in the fight against cancer. J Cancer Res Clin Oncol. 2019; 145(4): 797-810.
18. Zhou Y, Xu H, Wang H, Ye B C. Detection of breast cancer-derived exosomes using the horseradish peroxidase-mimicking DNAzyme as an aptasensor. Analyst. 2019; 145(1):107-114.
19. Zou J, Shi M, Liu X, Jin C, Xing X, Qiu L, Tan W. Aptamer-Functionalized Exosomes: Elucidating the Cellular Uptake Mechanism and the Potential for Cancer-Targeted Chemotherapy. Anal Chem 2019; 91 (3): 2425-2430.
20. Park S M, Aalipour A, Vermesh O, Yu J H, Gambhir S S. Towards clinically translatable in vivo nanodiagnostics. Nat Rev Mater. 2017; 2(5): 17014.
21. Jafari S H, Saadatpour Z, Salmaninejad A, Momeni F, Mokhtari M, Nahand J S, et al. Breast cancer diagnosis: Imaging techniques and biochemical markers. J Cell Physiol. 2018; 233(7): 5200-13.
22. Zhang K, Yue Y, Wu S, Liu W, Shi J, Zhang Z. Rapid Capture and Nondestructive Release of Extracellular Vesicles Using Aptamer-Based Magnetic Isolation. ACS Sens. 2019; 4(5): 1245-51.
23. Wang L, Pan Y, Liu Y, Sun Z, Huang Y, Li J, et al. Fabrication of an Aptamer-Coated Liposome Complex for the Detection and Profiling of Exosomes Based on Terminal Deoxynucleotidyl Transferase-Mediated Signal Amplification. ACS Appl Mater Interfaces. 2020; 12(1): 322-9.
24. Xie X, Nie H, Zhou Y, Lian S, Mei H, Lu Y, et al. Eliminating blood oncogenic exosomes into the small intestine with aptamer-functionalized nanoparticles. Nat Commun. 2019; 10(1):5476.
25. Quintavalle C. et al.: “Aptamer-mediated exosomes detection for early breast cancer identification”, Annals of Oncology, vol. 30, no. suppl 5 Oct. 2019, page v804
26. Ana Diaz-Fernández, Ramón Lorenzo-Gómez, Rebeca Miranda-Castro, Noemí de-Los-Santos-Álvarez, María Jesús Lobo-Castańón. Electrochemical aptasensors for cancer diagnosis in biological fluids—A review. Review Anal Chim Acta, 2020 Aug. 8; 1124:1-19. doi: 10.1016/j.aca.2020.04.022.
27. Yu An, Rui Li, Fan Zhang, Pingang He. Magneto-Mediated Electrochemical Sensor for Simultaneous Analysis of Breast Cancer Exosomal Proteins Anal Chem 2020 Apr. 7; 92(7):5404-5410. doi: 10.1021/acs.analchem.0c00106.
28. Hassn Eman M. et al. «Recent advances in cancer early detection and diagnosis: role of nucleic acid based aptasensors”, Trac Trends in analytical chemistry, vol. 124, 9 Jan. 2020. Doi:10.1016/j.trac.2020.115806
29. Rongrong Huang, Lei He, Yanyan Xia, Hongpan Xu, Chang Liu, Hui Xie, Su Wang, Lijun Peng, Yufeng Liu, Yuan Liu, Nongyue He, Zhiyang Li. A Sensitive Aptasensor Based on a Hemin/G-Quadruplex-Assisted Signal Amplification Strategy for Electrochemical Detection of Gastric Cancer Exosomes. Small 2019 May; 15(19):e1900735. doi: 10.1002/smll.201900735.
30. Tassilo Hornung, Heather A O'Neill, Stephen C Logie, Kimberly M Fowler, Janet E Duncan, Matthew Rosenow, Aniket S Bondre, Teresa Tinder, Varun Maher, Jelena Zarkovic, Zenyu Zhong, Melissa N Richards, Xixi Wei, Mark R Miglarese, Gunter Mayer, Michael Famulok, David Spetzler. ADAPT identifies an ESCRT complex composition that discriminates VCaP from LNCaP prostate cancer cell exosomes Nucleic Acids Res. 2020 May 7; 48(8):4013-4027. doi: 10.1093/nar/gkaa034.31.
31. Yifan Liu, Guang Chen, Dihua Shangguan, Liqin Zhang, Shuo Wan, Yuan Wu, Hui Zhang, Lian Duan, Chao Liu, Mingxu You, Jie Wang, Weihong Tan. Generating Cell Targeting Aptamers for Nanotheranostics Using Cell-SELEX. Theranostics. 2016 Jun. 15; 6(9):1440-52. doi: 10.7150/thno.15666.

1. A nucleotide aptamer or a variant thereof, wherein said aptamer comprises the sequence:
(SEQ ID No. 1)
5′ GGGAAGAGAAGGACAUAUGAUCGUGAUAGCUGUGGCAGUUAAGAAU
AGAUCUUCGCUGCGAUUGACUAGUACAUGACCACUUGA 3′
in which the underlined sequence
(SEQ ID No. 2)
CGUGAUAGCUGUGGCAGUUAAGAAUAGAUCUUCGCUGCGA
can be replaced with
(SEQ ID No. 3)
UGGCGGCUCGUUGAAGGUCGUCUCACGGGCUAGGAAUGCG
or with
(SEQ ID No. 4)
ACGGCUGUUGCUUCGAAUGAACCAAGGUUCCUCGGCGCAA
or
a functional fragment thereof in which said
fragment comprises the sequence
(SEQ ID No. 2)
CGUGAUAGCUGUGGCAGUUAAGAAUAGAUCUUCGCUGCGA
or
(SEQ ID No. 3)
UGGCGGCUCGUUGAAGGUCGUCUCACGGGCUAGGAAUGCG
or
(SEQ ID No. 4)
ACGGCUGUUGCUUCGAAUGAACCAAGGUUCCUCGGCGCAA.

2. The aptamer or variant thereof or a functional fragment thereof according to claim 1, wherein pyrimidine residues are replaced with 2′-fluoropyrimidine or wherein said aptamer or variant thereof or a functional fragment thereof is conjugated to at least one anticancer agent.

3. The aptamer or variant thereof or a functional fragment thereof according to claim 1 which comprises or consists of the sequence
(SEQ ID No. 9)
5′ UGUGGCAGUUAAGAAUAGAUCUUCGCUGCGAUU 3′.

4. (canceled)

5. A method for the treatment and/or prevention of a tumor comprising administering the aptamer or variant thereof or a functional fragment thereof of claim 1 to a patient in need thereof.

6. A method for selecting a nucleotide aptamer or a variant thereof comprising the steps of:
a) incubating a library of synthetic nucleotide oligomers with exosomes isolated from control cells, allowing the oligomers to bind thereto;
b) recovering a first series of nucleotide oligomers not bound to the exosomes isolated from control cells;
c) incubating said first series of nucleotide oligomers with exosomes isolated from target cells, allowing the first series of nucleotide oligomers to bind thereto;
d) recovering a second series of nucleotide oligomers bound to exosomes isolated from target cells;
e) amplifying said second series of nucleotide oligomers;
f) repeating steps a), b), c) and d) at least once and g) selecting and sequencing nucleotide oligomers bound to exosomes isolated from target cells;
wherein said nucleotide aptamer or a variant thereof binds specifically to exosomes isolated from target cells.

7. The method according to claim 6, wherein the synthetic nucleotide oligomers are labelled.

8. The method according to claim 6, wherein the nucleotide oligomers are oligoribonucleotides or modified nuclease-resistant oligoribonucleotides.

9. The method according to claim 6, wherein the exosomes are conjugated to magnetic beads.

10. The method according to claim 6, wherein the target cell is a tumor cell, or a breast cancer cell.

11. The method according to claim 6, wherein the library used in step a) is a complex library, or a library comprising at least 10¹⁴ oligonucleotides.

12. The method according to claim 6, wherein the library used in step a) is a RNA library prepared from the corresponding DNA library.

13. The method according to claim 6, wherein the library used in step a) is a RNA library comprising a pool of RNAs modified with 2′Fluoro-pyrimidines (2′F-Py).

14. The method according to claim 6, wherein in step a) oligomers bind to exosomes in a time comprised between 5 and 30 minutes.

15. The method according to claim 6, wherein said exosomes are whole exosomes.

16. The method according to claim 6, wherein said selected nucleotide aptamer or a variant thereof binds specifically to surface antigens of said exosomes isolated from target cells.

17. A nucleotide aptamer or a variant thereof obtainable by the method according to claim 6.

18. A pharmaceutical composition comprising the aptamer or the variant thereof of claim 1, said composition optionally further comprising another therapeutic agent.

19. A method for diagnosing a tumor in a patient from whom a sample was obtained, comprising:
incubating the sample with the aptamer or the variant thereof according to claim 1;
measuring the binding of the aptamer to the sample,
wherein said sample is optionally a blood, serum or saliva sample, a biopsy, urine or cerebrospinal fluid.

20. A kit for diagnosing a tumor comprising a nucleotide aptamer or the variant thereof according to claim 1.

21. A nucleic acid coding for the aptamer or the variant thereof according to claim 1.


SEQUENCE LISTING (10 sequences)

SEQ ID NO: 1 - DNA, 84 bp, Artificial Sequence
  synthetic
gggaagagaaggacauaugaucgugauagcuguggcaguuaagaauagaucuucgcugcgauugacuaguacaugaccacuuga

SEQ ID NO: 2 - DNA, 40 bp, Artificial Sequence
  synthetic
cgugauagcuguggcaguuaagaauagaucuucgcugcga

SEQ ID NO: 3 - DNA, 40 bp, Artificial Sequence
  synthetic
uggcggcucguugaaggucgucucacgggcuaggaaugcg

SEQ ID NO: 4 - DNA, 40 bp, Artificial Sequence
  synthetic
acggcuguugcuucgaaugaaccaagguuccucggcgcaa

SEQ ID NO: 5 - DNA, 44 bp, Artificial Sequence
  synthetic
ttcaggtaatacgactcactatagggaagagaaggacatatgat

SEQ ID NO: 6 - DNA, 23 bp, Artificial Sequence
  synthetic
tcaagtggtcatgtactagtcaa

SEQ ID NO: 7 - DNA, 84 bp, Artificial Sequence
  synthetic
gggaagagaaggacauaugauuggcggcucguugaaggucgucucacgggcuaggaaugcguugacuaguacaugaccacuuga

SEQ ID NO: 8 - DNA, 84 bp, Artificial Sequence
  synthetic
gggaagagaaggacauaugauacggcuguugcuucgaaugaaccaagguuccucggcgcaauugacuaguacaugaccacuuga

SEQ ID NO: 9 - DNA, 33 bp, Artificial Sequence
  synthetic
uguggcaguuaagaauagaucuucgcugcgauu

SEQ ID NO: 10 - DNA, 39 bp, Artificial Sequence
  synthetic
gccuuaguaacgugcuuugaugucgauucgacaggaggc